(19) United States
(12) Patent Application Publication
(10) Pub. No.: US 2017/0306411 A1
(43) Pub. Date: Oct. 26, 2017

(54) High-performance Method of Detecting mRNA Marker

(71) Applicant: Carygene International Biotechnology CO., LTD., Kaohsiung City (TW)

(72) Inventors: Chang-Han Wu, Kaohsiung (TW); Fu-Yen Chung, Kaohsiung (TW); Shiu-Ru Lin, Kaohsiung (TW); Chi-Kai Yang, Kaohsiung (TW)

(21) Appl. No.: 15/484,263

(22) Filed: Apr. 11, 2017

(30) Foreign Application Priority Data
Apr. 22, 2016 (TW) 105112584

(51) Int. Cl. C12Q 1/68

(52) U.S. Cl. CPC C12Q 1/6886; C12Q 2600/158

(57) ABSTRACT

The present invention improves the material of a CRC detection chip and the gene labeling, hybridization, reaction formula, and reaction time. Thus, physicians can clinically track and assess treatment plans for CRC patients and for various further treatments. The sensitivity and specificity of the CRC detection are improved with higher effectiveness than the traditional CEA detection.

### TECHNICAL FIELD OF THE INVENTION

The present invention relates to detecting mRNA marker; more particularly, relates to improving the material of a CRC detection chip and the gene labeling, hybridization, reaction formula, and reaction time for helping physicians to clinically track and assess treatment plans for colorectal cancer (CRC) patients and for various further treatments.

### DESCRIPTION OF THE RELATED ART(S)

For the past 30 years, the CRC patients of the United States with five-year survival has been raised from 50% to 65%. Yet, the International Union Against Cancer (UICC) announced that 30% to 40% of the second- and third-phase patients who received lesion resection will still relapse or die. Hence, the actual survival patients are still few, which means their relapses are early ones. Therefore, looking for early relapse diagnosis with high sensitivity and specificity is important.

For decades, tumor invasion depth, lymph node metastasis, and tip transfer are the main predictive modes proclaimed by the American Joint Committee on Cancer/International Union Against Cancer (AJCC/UICC) for forecasting patient's relapse after surgery. The development of early relapse diagnosis can effectively predict relapse of a patient after treatment. In 2009, the research report revealed by Nannini et al. pointed out a specific molecular CRC marker and a disease monitoring method for effectively improving the course of diagnosis and follow-up of early relapse. It can be found from the previous study that the undetected micrometastasis may make the CRC surgery fail. In 2010, Rahbari et al. suggested that the isolated tumor cells (ITC) could be used for early relapse prediction for the CRC patients after treatment. The early relapses of CRC patients are mainly caused by extreme malignant tumors (such as poor genotypes, tumor invasion depth, lymph node metastasis and terminal cancer) and ineffective chemotherapy. In the cases of early relapse, the survival rate is always low, so the development of early postoperative predictor is extremely valuable. However, some one- to three-phase patients still have metastases under standard treatments. Therefore, it is necessary to develop a way to improve early prediction for patients after treatment. Unfortunately, there is no effective way to distinguish between early-relapsed and non-relapsed patients.

Hence, the prior arts do not fulfill all users' requests on actual use.

### SUMMARY OF THE INVENTION

The main purpose of the present invention is to improve the material of a CRC detection chip and the gene labeling, hybridization, reaction formula, and reaction time for helping physicians to clinically track and assess treatment plans for CRC patients and for various further treatments, where the sensitivity and specificity of the CRC detection are improved with higher effectiveness than the traditional CEA detection.

To achieve the above purpose, the present invention is a high-performance method of detecting an mRNA marker, comprising steps of: (a) dot-blotting an oligonucleotide on a thermoplastic composite through a blotting device; placing the thermoplastic composite blotted with the oligonucleotide in a sterile drying oven to be hot-dried for 1.5˜2.5 hours (hrs) followed by UV irradiation and a fixing process to obtain an oligonucleotide chip, where the oligonucleotide contains a variety of target genes; and the oligonucleotide chip is formed with the thermoplastic composite covered by an mRNA sequence-specific oligonucleotide (SSO); (b) processing a test specimen through a pretreatment of cell lysis and RNA extraction to obtain a solution containing RNA (i.e. RNA solution) while DNA is removed; (c) adding a biotin-labeling solution to the RNA solution to process biotin-labeling at a temperature of 30˜45 celsius degrees (° C.) for 1.5˜2.5 hrs; then, adding the biotin-labeling solution repeatedly to reverse-transcribe cDNA by mRNA in a shaking water bath at a temperature of 30˜45° C. for 0.5˜1.5 hrs to obtain a cDNA solution; then, heating the cDNA solution at a temperature of 75˜115° C. for 4˜6 minutes; (d) directly reacting the cDNA solution with the oligonucleotide chip in an oven at a temperature of 34˜50° C. for 1.5˜2.5 hrs; then, adding a polyethylene glycol (PEG 6000) solution to complete hybridization at a temperature of 34˜50° C. for 1.5˜2.5 hrs; and (e) after completing the hybridization, adding a strep-avidin alkaline phosphatase (AP) solution to the oligonucleotide chip to process coloring with nitroblue tetrazolium (NBT) or 5-Bromo-4-chloro-3-indolyl-phosphate (BCIP) until mRNA of the test specimen shows. Accordingly, a novel high-performance method of detecting an mRNA marker is obtained

### BRIEF DESCRIPTION OF THE DRAWINGS

The present invention will be better understood from the following detailed description of the preferred embodiment according to the present invention, taken in conjunction with the accompanying drawings, in which

FIG. 1 is the flow view showing the preferred embodiment according to the present invention;

FIG. 2 is the view showing the relationship between the CRC-chip detection and the relapsed case; and

FIG. 3 is the view showing the early relapse diagnosis with the CRC detection chip and through the conventional carcinoembryonic antigen (CEA).

### DESCRIPTION OF THE PREFERRED EMBODIMENT

The following description of the preferred embodiment is provided to understand the features and the structures of the present invention.

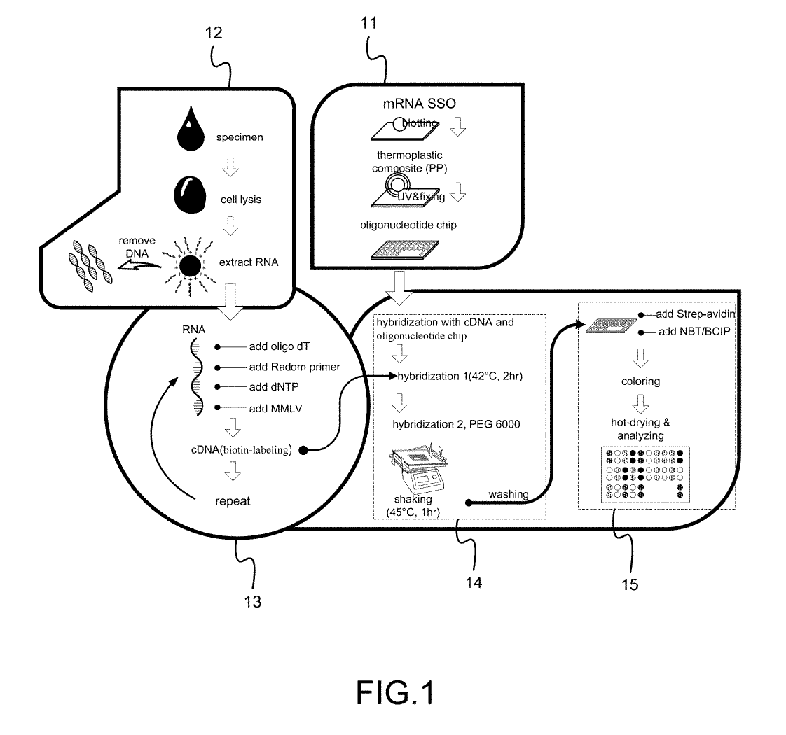

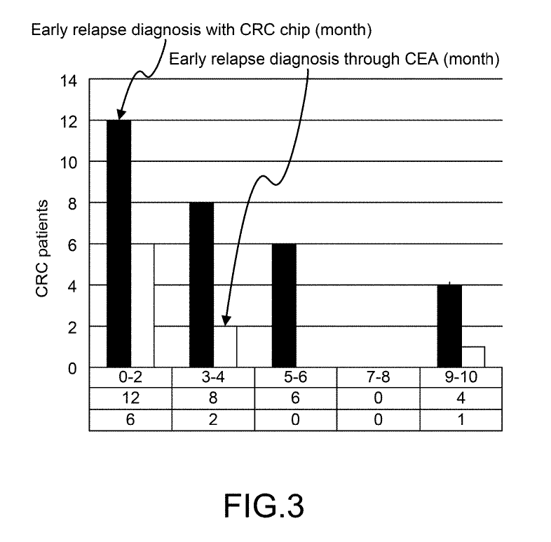

Please refer to FIG. 1˜FIG. 3, which are a flow view showing a preferred embodiment according to the present invention; a view showing a relationship between CRC-chip detection and relapsed case; and a view showing early relapse diagnosis with CRC detection chip and through conventional CEA. As shown in the figures, the present invention is a high-performance method of detecting an mRNA marker, comprising the following steps:

(a) Obtaining oligonucleotide chip 11: An oligonucleotide containing a variety of target genes are dot-blotted on a thermoplastic composite, e.g. polypropylene (PP), through a blotting device. Then the thermoplastic composite blotted with the oligonucleotide is placed in a sterile drying oven to be hot-dried for 2 hours (hrs) followed by UV irradiation and a fixing process. Thus, an oligonucleotide chip is obtained with the thermoplastic composite covered by an mRNA sequence-specific oligonucleotide (SSO).

(b) Obtaining RNA solution 12: A test specimen is processed through a pretreatment of cell lysis and RNA extraction. The cell lysis is a process of cell disruption repeated several times to obtain a cell exudate. The process of cell disruption is to shatter the test specimen through sonication; or to quickly freeze the test specimen with liquid nitrogen and immediately put the test specimen in a bath to be thawed at a temperature of 42-celsius degrees (° C.). The RNA extraction of the pretreatment comprises the following steps:
(b1) The cell exudate and a solution of proteinase K and guanidine thiocyanate are uniformly mixed at a ratio of 4:1.
(b2) After placing the mixed solution at a temperature of 37° C. for 1 hour (hr), a plurality of magnetic beads having positive electricity is added to process reaction in a shaking water bath for 30 minutes until nucleic acid of the test specimen is completely adsorbed on the magnetic beads.
(b3) A tube containing the mixed solution obtained after the reaction is placed on a bead seat to fix the magnetic beads at bottom of the tube.
(b4) The mixed solution is sucked out but the magnetic beads are left and the magnetic beads are washed with absolute alcohol for three times.
(b5) The nucleic acid adsorbed on the magnetic beads is eluted with a tris-EDTA (TE) buffer to obtain a nucleic acid solution.
(b6) A DNA enzyme is added to the nucleic acid solution to process reaction at a temperature of 37° C. for 15 minutes. Then, the nucleic acid solution is heated to a temperature of 95° C. for 5 minutes to remove DNA and obtain a solution containing RNA (i.e. RNA solution).

(c) Labeling biotin 13: A biotin-labeling solution is added to the RNA solution to process biotin-labeling at a temperature of 37° C. for 2 hrs, where the biotin-labeling solution is a solution having a labeling group consisting of oligo-dt-anchor primer, random hexamer, deoxyribonucleoside triphosphate (dNTP), biotinylated-deoxyuridine triphosphate (biotin-dUTP), Moloney murine leukemia virus (MMLV) reverse transcriptase (RTase), and a ribonuclease (RNAse) inhibitor. Then, the biotin-labeling solution is added repeatedly to reverse-transcribe cDNA by mRNA in a shaking water bath at a temperature of 37° C. for 1 hr until finishing biotin-labeling. Then, a cDNA solution is obtained to be heated at a temperature of 95° C. for 5 minutes after finishing the biotin-labeling.

(d) Processing hybridization 14: The cDNA solution is directly reacted with the oligonucleotide chip in an oven at a temperature of 42° C. for 2 hrs. Then, a polyethylene glycol (PEG 6000) solution is added to complete hybridization at a temperature of 45° C. for 1 hr.

(e) Showing mRNA 15: After the hybridization is completed, the oligonucleotide chip is washed with a cleaning solution and added with a strep-avidin alkaline phosphatase (AP) solution to process coloring with nitroblue tetrazolium (NBT) or 5-Bromo-4-chloro-3-indolyl-phosphate (BCIP) until mRNA of the test specimen shows.

Through the above steps, a colorectal cancer (CRC) detection chip can be obtained as a highly-efficient detection platform for labeling mRNA.

In a state-of-use, all samples of clinical patients are collected from the same medical center by the same surgical team during June 2015 and March 2016. Tracking work is mainly done according to the clinical practice guideline (CPG) of the European Society for Medical Oncology (ESMO). Postoperative monitoring items include patient medical history, health examination and clinical tracking project. Every patient has to do ultrasonography or computed tomography (CT) once per month and to take a chest plain film per three months. Any CRC patient has new or metastatic lesion after treatment is defined as relapsed. Tracking time ends when the patient dies or on Apr. 4, 2016.

On using the present invention, a detection following the above steps is processed to obtain a detection result for statistical analysis through SPSS version 14.0. All data are presented as mean±SD (SD=standard deviation). Two treatment results, including a radiation treatment result and a chemotherapy result, and their gene expressions are analyzed through chi-square test. If the analysis result is P<0.05, there is a significant difference.

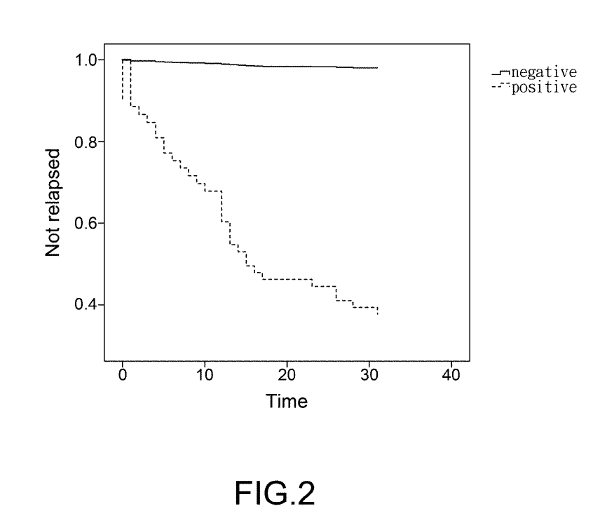

A total of 253 CRC patient's samples is collected during June 2015 to March 2016, where 34 patients have an early relapse. The patients without relapse include 120 males and 99 females with an average age of 64.5±11.6 years old; the patients with relapse includes 17 males and 17 females with an average age of 66.6±11.7 years old. The result is shown in Table 1, which shows a CEA positive result (≧5 ng/mL) and a CRC-chip positive result and a significant difference (P=0.012; P<0.0001) in between. In the relationship analysis between the CRC-chip positive result and the relapsed cases, the Cox-regression analysis is used. As shown in FIG. 2, the patients having positive reactions to the CRC detection chips have higher relapse rates than those having negative reactions. In addition, as shown in Table 2, through a statistical analysis, the CEA detection and the CRC-chip detection show 26.47% and 88.24% as well as 89.04% and 91.78% for sensitivity and specificity on predicting CRC relapse, respectively. Therein, column LR+ shows positive likelihood ratio; column LR− shows negative likelihood ratio; and column CI shows confidence interval.

TABLE 1

| | Not relapsed | Relapsed | |
|---|---|---|---|
| Variable | (n = 221) | (n = 34) | P value |
| Age (yrs) | 64.5 ± 11.6 | 66.6 ± 11.7 | 0.321 |
| (Mean ± SD) | | | |
| Gender | | | |
| Male | 120 | 17 | 0.602 |
| Female | 99 | 17 | |
| TNM stage | | | |
| I | 54 | 5 | 0.264 |
| II | 112 | 17 | |
| III | 53 | 12 | |
| CEA (ng/mL) | | | |
| <5 | 195 | 25 | 0.012 |
| ≧5 | 24 | 9 | |
| CRC chip | | | |
| Negative | 201 | 4 | <0.0001 |
| Positive | 18 | 30 | |

*TNM = lymph node transfer

TABLE 2

| | Sensitivity | Specificity | | |
|---|---|---|---|---|
| | (%) | (%) | LR+ | LR− |
| CEA | 26.47 | 89.04 | 2.415 | 0.826 |
| (95% CI) | (14.60- | (84.21- | (1.229- | (0.671- |
| | 43.12) | 92.52) | 4.747) | 1.016) |
| CRC chip | 88.24 | 91.78 | 10.735 | 0.128 |
| (95% CI) | (73.38- | (87.38- | (6.782- | (0.051- |
| | 95.33) | 94.74) | 16.993) | 0.322) |

Besides, as shown in FIG.3, the CRC-chip detection can detect the relapse of CRC patient earlier than the traditional CEA detection.

As shown above, the result obtained for predicting CRC relapse by using the CEA and CRC-chip detection shows that the sensitivity and specificity of the CRC-chip detection for predicting CRC relapse are significantly higher than those of the CEA detection. Thus, it is confirmed that the CRC detection chip fabricated according to the present invention has the potential to be an effective tool for predicting CRC relapse.

To sum up, the present invention is a high-performance method of detecting an mRNA marker, where a CRC detection chip of oligonucleotide containing a variety of target genes is used for detection to find 5 circulating tumor cells (CTC) per 1 milliliter in the peripheral blood of a CRC patients (approximately 1 tumor cell in every 106 white blood cells); and, by improving the material of the chip and the gene labeling, hybridization, reaction formula, and reaction time, not only sensitivity and specificity are effectively improved, but also the chip can be used as a simple detection tool for helping physicians to clinically track and assess treatment plans for CRC patients and for various further treatments.

The preferred embodiment herein disclosed is not intended to unnecessarily limit the scope of the invention. Therefore, simple modifications or variations belonging to the equivalent of the scope of the claims and the instructions disclosed herein for a patent are all within the scope of the present invention.

what is claimed is:

1. A method of detecting mRNA marker, comprising steps of:
(a) dot-blotting an oligonucleotide on a thermoplastic composite through a blotting device; placing said thermoplastic composite blotted with said oligonucleotide in a sterile drying oven to be hot-dried for 1.518 2.5 hours (hrs) followed by UV irradiation and a fixing process to obtain an oligonucleotide chip,
wherein said oligonucleotide contains a variety of target genes; and said oligonucleotide chip is obtained with said thermoplastic composite covered by an mRNA sequence-specific oligonucleotide (SSO);
(b) processing a test specimen through a pretreatment of cell lysis and RNA extraction to obtain a solution containing RNA (i.e. RNA solution) while DNA is removed;
(c) adding a biotin-labeling solution to said RNA solution to process biotin-labeling at a temperature of 30˜45 celsius degrees (° C.) for 1.5˜2.5 hrs; then, adding said biotin-labeling solution repeatedly to reverse-transcribe cDNA by mRNA in a shaking water bath at a temperature of 30˜45° C. for 0.5˜1.5 hrs to obtain a cDNA solution; then, heating said cDNA solution at a temperature of 75˜115° C. for 4˜6 minutes;
(d) directly reacting said cDNA solution with said oligonucleotide chip in an oven at a temperature of 34˜50° C. for 1.5˜2.5 hrs; then, adding a polyethylene glycol (PEG 6000) solution to complete hybridization at a temperature of 34˜50° C. for 1.5˜2.5 hrs; and
(e) after completing said hybridization, adding a strep-avidin alkaline phosphatase (AP) solution to said oligonucleotide chip to process coloring with a material selected from a group consisting of nitroblue tetrazolium (NBT) and 5-Bromo-4-chloro-3-indolyl-phosphate (BCIP) until mRNA of said test specimen shows.

2. The method according to claim 1,
wherein said thermoplastic composite is polypropylene (PP).

3. The method according to claim 1,
wherein said oligonucleotide chip detects circulating tumor cells (CTCs) in 5 cells per milliliter (ml) of peripheral blood of a colorectal cancer patient.

4. The method according to claim 1,
wherein, in step (b), said cell lysis is a process of cell disruption repeated several times to obtain a cell exudate and said process is selected from a group consisting of shattering said test specimen through sonication; and
quickly-freezing said test specimen with liquid nitrogen and immediately putting said test specimen in a bath to be thawed at a temperature of 3550° C.

5. The method according to claim 1, wherein, in step (b), said RNA extraction comprises steps of:
(b1) uniformly mixing a cell exudate and a solution at a ratio of 4±20%:1±20% to obtain a mixed solution, said cell exudate being obtained by said cell lysis, said solution being a solution of proteinase K and guanidine thiocyanate;
(b2) after placing said mixed solution at a temperature of 30˜45° C. for 0.5˜1.5 hours (hrs), adding a plurality of magnetic beads having positive electricity to process reaction in a shaking water bath for 24˜36 minutes until nucleic acid of said test specimen is completely adsorbed on said magnetic beads;
(b3) placing a tube containing said mixed solution on a bead seat to fix said magnetic beads at bottom of said tube;
(b4) sucking out said mixed solution but leaving said magnetic beads and washing said magnetic beads with absolute alcohol for three times;
(b5) eluting said nucleic acid adsorbed on said magnetic beads with a tris-EDTA (TE) buffer to obtain a nucleic acid solution and process reaction with a DNA enzyme added;
(b6) after finishing reaction and staying said nucleic acid solution at a temperature of 30˜45° C. for 10˜20 minutes, heating said nucleic acid solution at a temperature of 75˜115° C. for 5 minutes to remove DNA and obtain said RNA solution.

6. The method according to claim 1,
wherein, in step (c), said biotin-labeling solution is a solution having a labeling group consisting of oligo-dt-anchor primer, random hexamer, deoxyribonucleoside triphosphate (dNTP), biotinylated-deoxyuridine triphosphate (biotin-dUTP), Moloney murine leukemia virus (MMLV) reverse transcriptase (RTase), and ribonuclease (RNAse) inhibitor.
